(19) United States
(12) Patent Application Publication
(10) Pub. No.: US 2009/0182151 A1
(43) Pub. Date: Jul. 16, 2009

(54) Method for making amphiphilic dendrimers

(75) Inventors: Peng Wu, Berkeley, CA (US); Valery V. Fokin, Oceanside, CA (US); K. Barry Sharpless, La Jolla, CA (US)

Correspondence Address: Olson & Cepuritis, LTD., 20 NORTH WACKER DRIVE, 36TH FLOOR, CHICAGO, IL, 60606, US

(73) Assignee: The Scripps Research Institute, La Jolla, CA (US)

(21) Appl. No.: 11/989,072

(22) PCT Filed: Jul. 18, 2006
(86) PCT No.: PCT/US2006/028017

§ 371 Date: Feb. 18, 2009

Related U.S. Application Data
(60) Provisional application No. 60/700,482, filed on Jul. 18, 2005.

(51) Int. Cl. C07D 403/14

(52) U.S. Cl. 548/255; 977/754

(57) ABSTRACT

A series of AB-type amphiphilic dendritic polyesters have been prepared divergently, in which two hybrids were coupled via the copper(1)-catalyzed triazole formation.

### TECHNICAL FIELD

The invention relates to dendrimers and to a method for making di-block dendrimers. More particularly, the invention relates to the use of click chemistry for making di-block dendrimers.

### BACKGROUND

Molecular amphiphiles have myriad application potentials, such as nanocarriers, (Joester, D., et al., Angew. Chem., Int. Ed. 2003, 42, 1486; and Stiriba, S. E., et al., Angew. Chem., Int. Ed. 2002, 41, 1329) structure directing agents for nanostructure formation, (Sone, E. D., et al., Angew. Chem., Int. Ed. 2002, 41, 1706; Zhao, D., et al., Science 1998, 279, 548; Cha, J. N., et al., Nature (London) 2000, 403, 289; Simon, P. F. W., et al., Chem. Mater. 2001, 12, 3464; Bagshaw, S. A., et al., Science 1995, 269, 1242; and Hartgerink, J. D., et al., Science 2001, 294, 1684) or as catalysts. (Piotti, M. E., et al., J. Am. Chem. Soc. 1999, 121, 9471; Hecht, S., et al., J. Am. Chem. Soc. 2001, 123, 6959; and Boerakker, M. J., et al., Angew. Chem., Int. Ed. 2002, 41, 4239) The unique properties possessed by these molecules, including fluidity and compartmentalization, rely on their amphiphilic nature driving the assembly and organization into tridimentional network. For example, a triblock amphiphilic copolymer has been developed by Nie and coworkers as the encapsulating tool of quantum dots (QD) for in vivo cancer imaging. (Gao, X., et al., Nat. Biotechnol. 2004, 22, 198) This polymer consists of a polybutylacrylate segment (hydrophobic), a polyethylacrylate segment (hydrophobic), a polymethacrylic acid segment (hydrophilic) and a hydrophobic hydrocarbon side chain. Through a spontaneous self-assembly process, the polymer can disperse and encapsulate single tri-n-octylphosphine oxide (TOPO)-capped QD, offering protection over a broad pH range and salt conditions.

Besides linear polymers, dendrimers with well-defined structures and monodispersity are attractive candidates for the construction of amphiphiles and self-assembling materials. Most amphiphilic dendrimers to date possess core-shell architectures with a combination of hydrophobic coils and hydrophilic poly(amidoamine) (PAMAM) or poly(propyleneimine) (PPI) in the branch. (Gitsov, I., et al., Macromolecules 1993, 26, 5621; Iyer, J., et al., Macromolecules 1998, 31, 8757; Iyer, J., et al., Langmuir 1999, 15, 1299; and Cameron, J. H., et al., Adv. Mater. 1997, 9, 398) Few reports have described dendrimers with wedge shaped regions tailored with hydrophilic and hydrophobic functionalities at the periphery. (Hawker, C. J., et al., J. Chem. Soc., Perkin Trans. 1 1993, 1287-1297) Only through the utilization of protecting groups have representative molecules of this type been prepared via the divergent synthetic approach, but these methodologies are not generally applicable. (Aoi, K., et al., Macromolecules 1997, 30, 8072; Maruo, N., et al., Chem. Commun. 1999, 2057-2058; and Pan, Y., et al., Macromolecules 1999, 32, 5468-5470) The convergent approach provides a more general way for the preparation of these segmented macromolecules. However, an excess of monomers has to be applied to control reactions at the two possible growth sites. (Grayson, S. M., et al., Chem. Rev. 2001, 101, 3919-3967)

What is needed is a method for synthesizing di-block amphiphilic dendrimers via a divergent approach. What is needed is a method is the use of copper(I)-catalyzed cycloaddition to couple two hybrids decorated with hydrophilic and hydrophobic peripheries.

### SUMMARY

A series of AB-type amphiphilic dendritic polyesters have been prepared divergently, in which two hybrids were coupled via the copper(I)-catalyzed triazole formation. The unique nature of this new class of dendrimers permitted the installation of different functionalities at the individual blocks sequentially. Our goal is to develop the resulting segmented macromolecules as bacterial detection tools. Carbohydrate ligands have been displayed on the periphery of block A, to allow for multivalent interaction with pathogens, such as Escherichia coli. Coumarin derivatives have been attached to block B, to allow for confocal microscopic visualization and flow cytometry quantification.

One aspect of the invention is directed to a process for making a di-block dendrimer. The di-block dendrimer is of a type having a first dendritic block and a second dendritic block. The first dendritic block has a first block core; the second dendritic block has a second block core. The process employs the step of coupling the first block core to the second block core by means of a click chemistry reaction to form the di-block dendrimer having a di-block core. In a preferred embodiment, the click chemistry reaction is a 1,3-dipolar cycloaddition of a terminal acetylene with an azide to form a [1,2,3]-triazole. The first block core may include a terminal acetylene and the second core block may include an azide. In another preferred mode, the first dendritic block includes a first periphery, the second dendritic block includes a second periphery, and the first periphery differs from the second periphery.

Another aspect of the invention is directed to an improved dendritic block having a block core characterized by having a terminal acetylene.

Another aspect of the invention is directed to an improved dendritic block having a block core characterized by having an azide.

Another aspect of the invention is directed to an improved di-block dendrimer having a first dendritic block, a second dendritic block, and a di-block core that couples the first dendritic block to the second dendritic block. In this embodiment, the di-block core is characterized by a [1,2,3]-triazole ring that couples the first dendritic block to the second dendritic block.

### BRIEF DESCRIPTION OF DRAWINGS

FIG. 1 illustrates a scheme for the synthetic strategy toward di-block amphiphilic dendrimers.

FIG. 2 illustrates a scheme for the synthesis of a dendritic di-block with hydrophilic (3.8) functional groups at the periphery and of a dendritic di-block with hydrophobic (3.4) at the periphery.

FIG. 3 illustrates a proton NMR spectrum for dendron (An)₈-[G-4]-acet (3.4). The resulting dendritic fragments gave distinctive peaks on the ¹H-NMR.

FIG. 4 illustrates a proton NMR spectrum for dendron (OH)₁₆-[G-4]-Az (3.8). The resulting dendritic fragments gave distinctive peaks on the ¹H-NMR.

FIG. 5 illustrates a reaction scheme for the synthesis of (An)₄-[G-3]-[G-3]-(OH)₈ (3.10).

FIG. 6 illustrates a MALDI spectrum of dendrimer (An)₄-[G-3]-[G-3]-(OH)₈ (3.10).

FIG. 7 illustrates a table characterizing the indicated dendrimers.

FIGS. 8a, 8b, and 8c illustrate a synthetic scheme for the postcycloaddition modification of amphiphilic dendrimer (An)₁₆-[G-4]-[G-1]-(OH)₂, (3.14).

### DETAILED DESCRIPTION

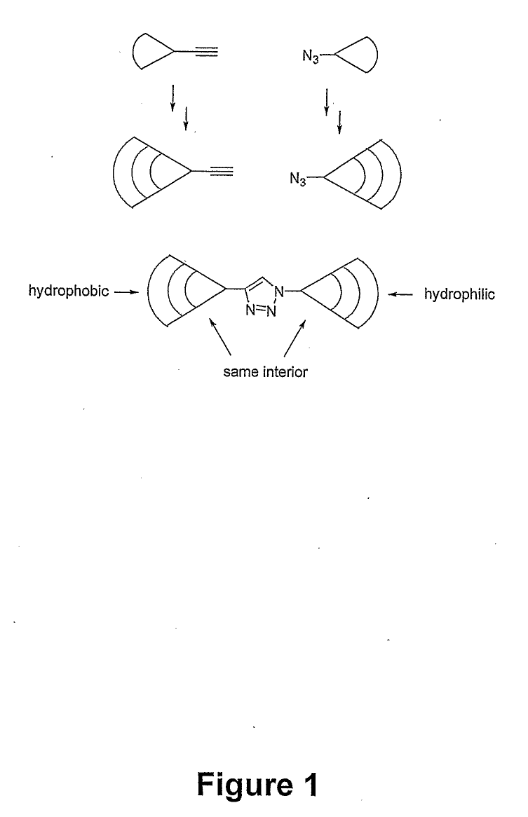

A divergent approach was employed in our dendrimer synthesis. As azides and acetylenes are nearly inert to a variety of chemical transformations, the introduction of both functionalities to the focal point was envisaged in the very beginning stage of the synthesis. Growth of the branches continued outward by iterative coupling and activation steps, furnishing higher generation dendritic segments with hydrophilic and hydrophobic groups at the periphery. In the final step, copper(I)-catalyzed cycloaddition joined the two segments together to form the desired amphiphilic dendrimers (FIG. 1).

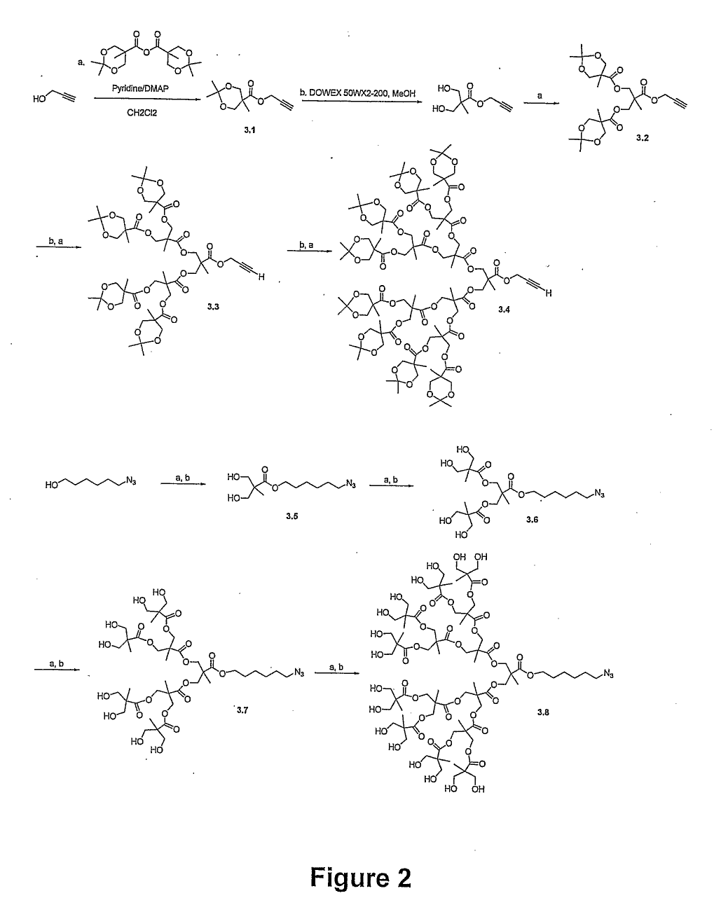

Azide and acetylene groups were introduced at the focal point by coupling the anhydride of isopropylidene-2,2-bis(methoxy)propionic acid with 6-azidohexanol and propargyl alcohol respectively (FIG. 2). After removing the acetonide-protecting group using DOWEX 50WX2-200 resin in methanol, the free hydroxyl groups were reacted with the anhydride using the method developed by Malkoch and Hult. (Malkoch, M., et al., Macromolecules 2002, 35, 8307-8314) The ratios of 5 equiv of pyridine, 0.15 equiv of DMAP, and 1.3 equiv of the anhydride to hydroxyl group gave the optimal results. After repeating the two-step deprotecting and coupling sequence, dendritic fragments with hydrophilic and hydrophobic end groups were obtained in high yield and purity up to the 4^{th} generation.

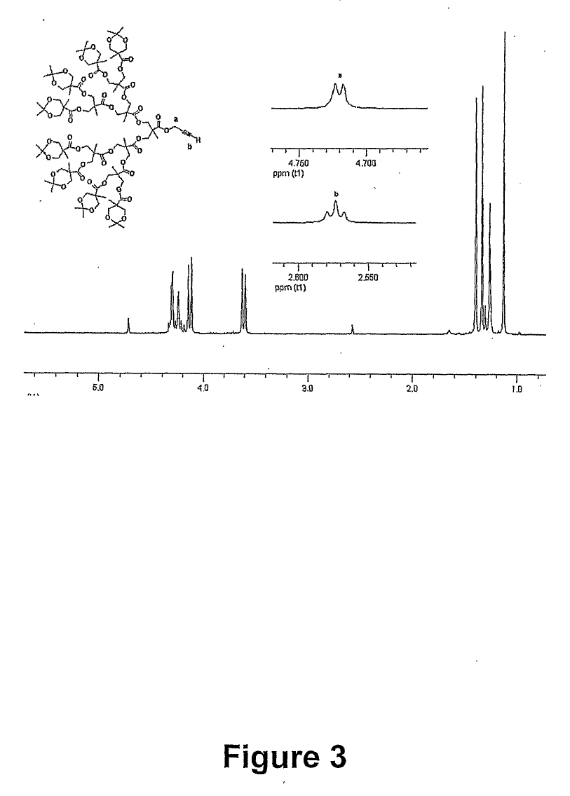

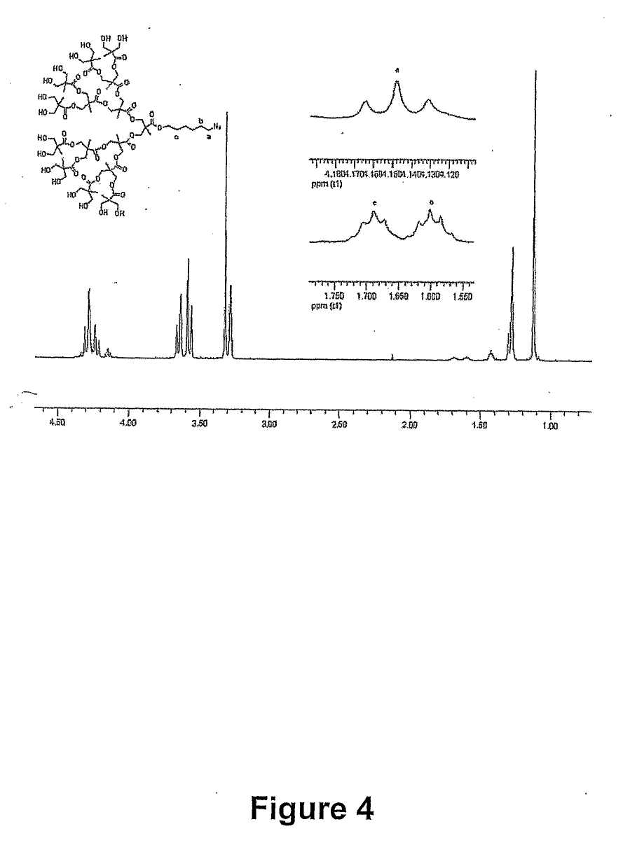

The resulting dendritic fragments gave distinctive peaks on the ¹H-NMR. The acetylinic proton appeared as a doublet at ca. 2.57 ppm, the propargylic —CH₂ as a sharp triplet at ca. 4.72 ppm and —CH₂N₃ as a sharp triplet at ca. 4.15 ppm (FIG. 3 and FIG. 4).

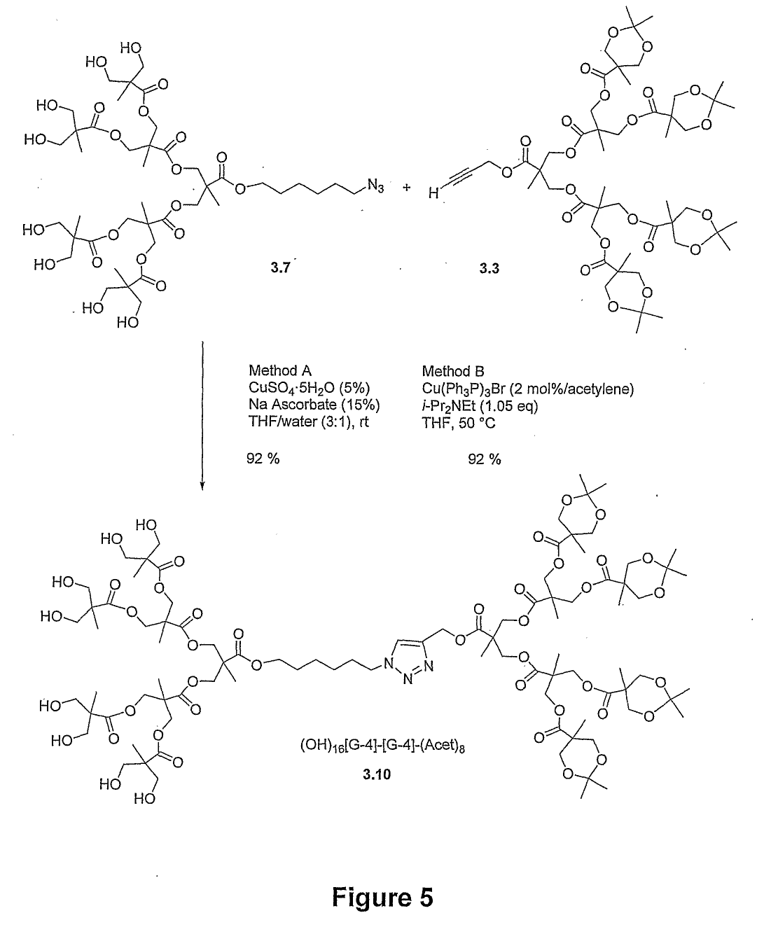

With both hemispherical dendrons in hand, the stage was set for the copper(I)-catalyzed cycloaddition to bring the two halves together. As a test experiment, (OH)₈-[G-3]-Az, 3.7, and (An)₄-[G-3]-Acet, 3.3, were mixed in THF/water (3:1) solution before the addition of CuSO₄.5H₂O (5 mol %) and sodium ascorbate (15 mol %) (method A, FIG. 5). 3.3 was used 2-5% in excess to ensure the full conversion. The reaction finished overnight as indicated by LC-MS analysis.

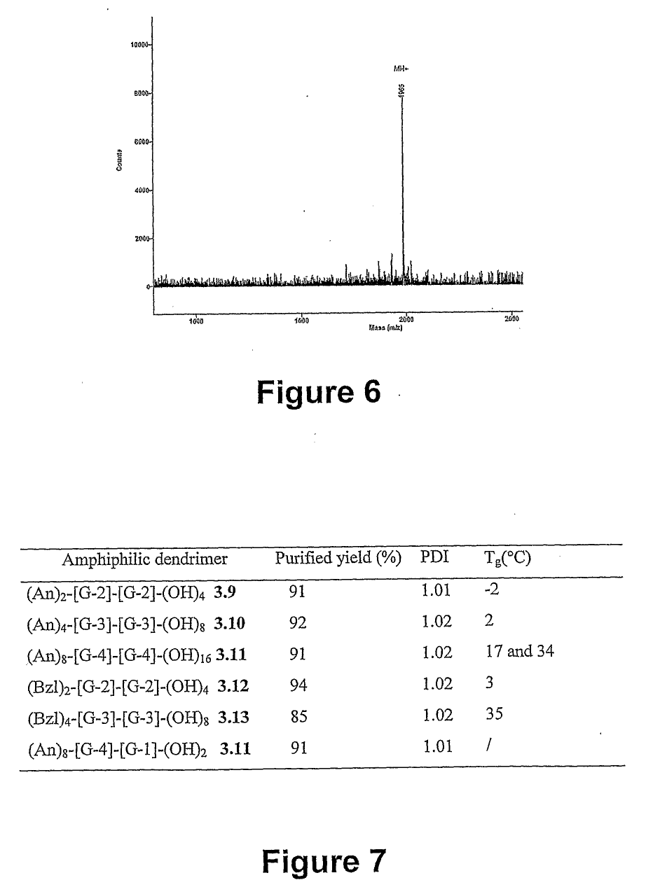

After purification by flash chromatography, analysis of the isolated product by MALDI-TOF indicated no presence of the azide and acetylene starting materials; formation of the product was confirmed by the appearance of a series of peaks at 1927, 1967 and 2007 (MNa⁺). Peaks at 1967 and 1927 corresponded to the removal of one and two acetonide protecting groups from the dendrimer due to its labile nature in aqueous solutions in the presence of trace amount of Lewis acidic copper(II). To overcome the incompatibility with aqueous conditions, the coupling was carried out in dry THF using [Cu(PPh₃)₃Br] as catalyst with N, N-diisopropylethylamine as the base (method B). 3.10 was isolated in 92% yield after removal of the catalyst and excess acetylene dendron by chromatography. MALDI analysis gave a single peak at 1985 (MH⁺), confirming the high efficiency of this transformation (FIG. 6). Using the same method, a series of amphiphilic dendrimers were prepared (FIG. 7). Replacing acetonide protecting groups with benzylidines resulted in dendrimers 3.12-3.13. Analysis of the dendrimers by MALDI-TOF mass spectrometry and gel-permeation chromatography (GPC) showed that the structures were monodisperse (FIG. 7).

Heating and cooling scans were performed at a rate of 10° C./min. 2^{nd} and 3^{rd} generation dendrimers showed a single T_{g}, which increased with molecular weight and generation. In the [G-4] case, large polarity differences drove the separation of the two phases and resulted in the observation of two T_{g}s (17° C. and 34° C.). These two glass transition temperatures are intermediates between the values for the two parent dendrons, 5° C. for (An)₈-[G-4]-Acet and 57° C. for (OH)₁₆-[G-4]-Az. (For examples of phase separation in dendritic block copolymers, see Hawker, C. J., et al., J. Chem. Soc., Perkin Trans. 11993, 1287-1297).

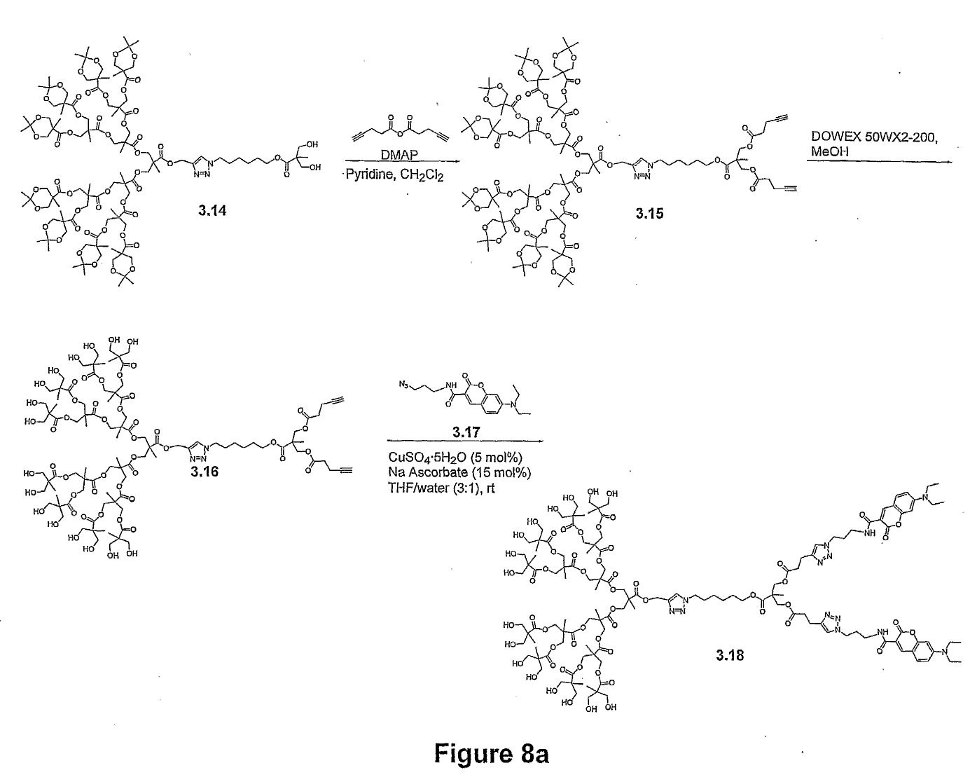

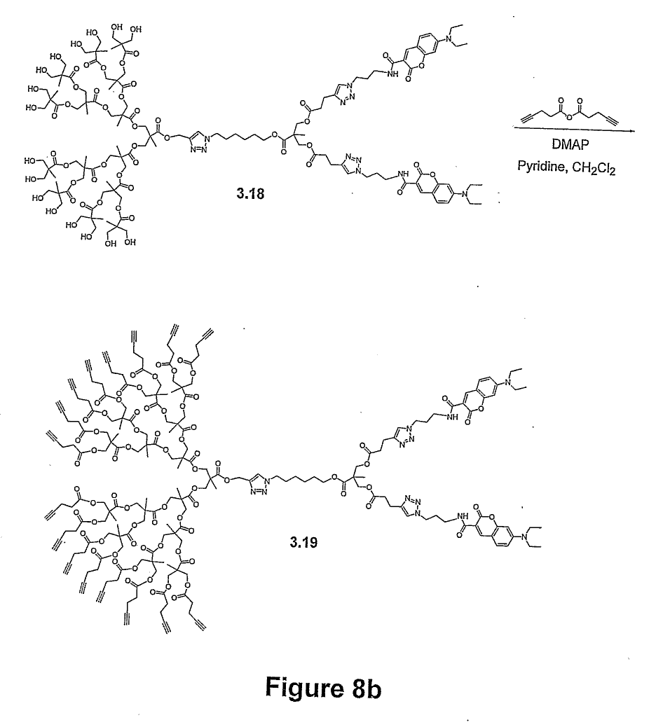

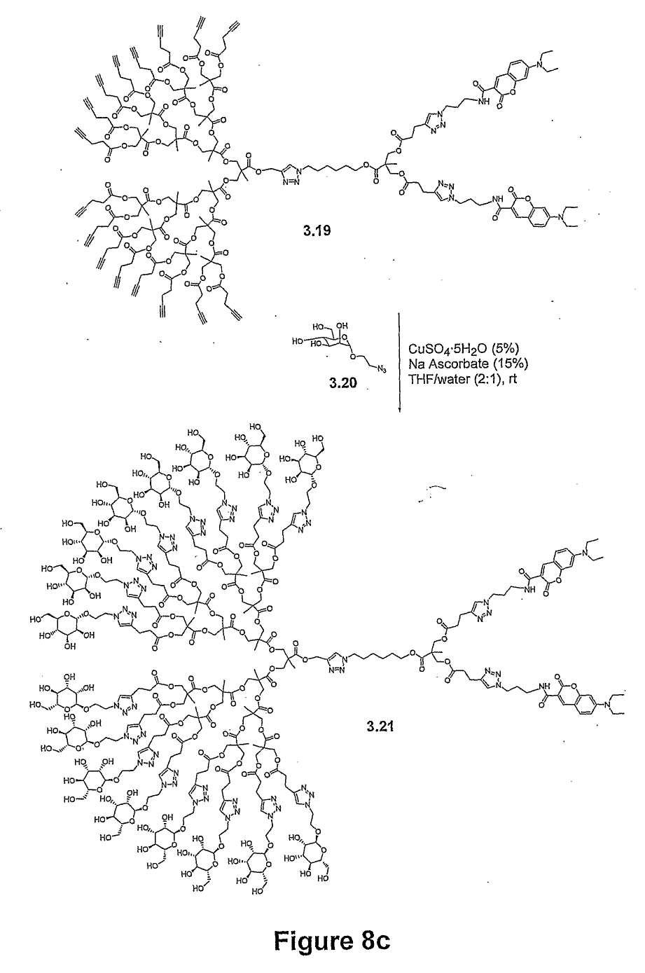

The unique nature of this new class of macromolecules permitted further modifications by introducing different functionalities at the periphery of individual blocks sequentially. As exemplified by the postcycloadditional modification of dendrimer (An)₁₆-[G-4]-[G-1]-(OH)₂, 3.14, acetylene groups were first introduced to the right hemisphere of the dendrimer by coupling the two hydroxyl groups with pent-4-ynoic anhydride (FIGS. 8A, 8B, and 8C). Removal of the acetonide protection groups on the left hemisphere gave dendrimer 3.16. 7-Diethylaminocoumarin based azide, 3.17, was then installed using method A to finish the right-hand functionalization. After incorporating 16 acetylenes at the left hemisphere, the resulting dendrimer was reacted with 2-azidoethyl-α-D-mannopyranoside 3.20 in THF/water mixture (method A) to furnish the carbohydrate coating. This bifunctional dendritic nano device is equipped with mannose as the multivalent binding agent for targeting of pathogens and coumarin as the detecting motif.

### EXPERIMENTAL

### General Methods

Analytical TLC was performed on commercial Merck Plates coated with silica gel GF254 (0.24 mm thick). Silica for flash chromatography was Merck Kieselgel 60 (230-400 mesh, ASTM). ¹H NMR (400 MHz) and ¹³C NMR (100 MHz) measurements were performed on a Bruker AC 400, 500 or 600 spectrometer at room temperature. Coupling constants (J) are reported in Hertz, and chemical shifts are reported in parts per million (δ) relative to CHCl₃ (7.26 for ¹H and 77.2 for ¹³C) or MeOD (3.31 for ¹H and 49.1 for ¹³C as internal reference. Size exclusion chromatography (SEC) was carried out at room temperature on a Waters chromatograph connected to a Waters 410 differential refractometer and six Waters Styragel® columns (five HR-5 μm and one HMW-20 μm) using THF as eluant (flow rate: 1 mL/min). A Waters 410 differential refractometer and a 996 photodiode array detector were employed. The molecular weights of the polymers were calculated relative to linear polystyrene standards. Non-aqueous copper(I)-catalyzed cycloaddition were performed in sealed tubes using a SmithCreator microwave reactor (Personal Chemistry Inc.). The modulated differential scanning calorimetry (MDSC) measurements were performed with a TA Instruments DSC 2920 and a ramp rate of 4 degrees per minute. The thermal gravimetric analysis measurements were done with a TA Instruments Hi-Res TGA 2950, under nitrogen purge, and the ramp rate was 10 degrees per minute. MALDI-TOF mass spectrometry was performed on a PerSeptive Biosystems Voyager DE mass spectrometer operating in linear mode, using dithranol in combination with silver trifluoroacetate as matrix. 3.17 (Zhu, L., et al., Tetrahedron 2004, 60, 7267-7275) and 3.20 (Arce, E., et al., Bioconjugate Chem. 2003, 14, 817-823) were synthesized as described previously.

### Nomenclature.

The nomenclature used for dendritic structures described in this chapter is as follows: (P)ₙ-[G-X]-F for dendrons, where P describes the external functional group, either OH for hydroxyl, An for acetonide, Bzl for benzylidene, Acet for acetylene; n indicates the number of chain end functionalities; X indicates the generation number of the dendritic framework and F describes the functional group at the focal point; either Acet for acetylene, or Az for azide. (P)ₙ-[G-X]-[G-X]-(P)ₙ for triazole linked amphiphilic dendrimers, P describes the external functional group, Cm stands for 7-Diethylaminocoumarin, Mann stands for α-D-mannopyranoside.

As employed herein the term “dendrimer” refers to polymers having a regular branched structure of a fractal nature. Dendrimers have a core from which the inner branches emanate. Further branches may emanate from the inner branches and so forth. Distal from the core are the terminal branches, i.e., branches from which no further branches emanate. The periphery is defined as that portion of the dendrimeric polymer attached to the distal branches from which no further branches emanate. The periphery consists of the collection of terminal chains, i.e., that portion of the dendrimeric polymer distal from the terminal branches and ending with the chain ends. As an inherent consequence of their fractal nature, dendrimers have a large number of functional groups at their chain ends. It is the chain ends that interact with the environment of the dendrimer and impart the properties of the dendrimer. The terms “chain end” and “functional group” are somewhat synonymous. However, the term “chain end” emphasizes the physical location of a section of the dendrimer; and the term “functional group” emphasizes the physical properties imparted by the “chain end”. The “functional group” may be any chemical moiety compatible for use as “chain end”.

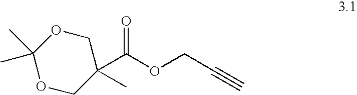

### General Procedure for the Dendritic Generation Growth Through Anhydride Coupling Reaction, (An)₁-[G-1]-Acet, 3.1.

Propargyl alcohol (10.0 g, 178 mmol) and DMAP (3.26 g, 26.7 mmol) were dissolved in pyridine (41.8 g, 535 mmol) in a 250 mL round bottom flask, followed by the addition of 100 mL CH₂Cl₂. The anhydride of isopropylidene-2,2-bis(methoxy)propionic acid (bis-MPA) (76.4 g, 231 mmol) was added slowly. The solution was stirred at room temperature overnight and monitored with ¹³C NMR until the reaction reached completion (determined by the presence of the excess anhydride at ˜169 ppm). The reaction was quenched with 5 mL of water under vigorous stirring, followed by dilution with 500 ml of CH₂Cl₂ and the solution was washed with 10% of NaHSO₄ (3×200 mL), and 10% of Na₂CO₃ (3×200 mL) and brine (100 mL). The organic phase was dried with MgSO₄, filtered, and concentrated. The crude product was purified by flash chromatography on silica, eluting with hexane (100 mL) and gradually increasing the polarity to EtOAc:hexane (10:90, 700 mL), followed by EtOAc:hexane (15:85) to give 3.1 as a colorless oil. Yield: 35.9 g (95%).

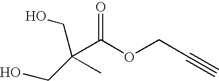

### General Deprotection Procedure of the Acetonide Group Using DOWEX 50W-X2-200 Resin, (HO)₂-[G-1]-Acet.

15 g DOWEX 50W-X2-200 resin were added to a solution of 6.1 (10.0 g, 47.1 mmol) in 300 mL of methanol in a 500 mL round bottom flask. The mixture was stirred at 40° C. and the deprotection was followed with ¹³C NMR until complete disappearance of peaks unique for the acetonide group was achieved, (i.e. the quaternary carbon at ˜98 ppm). The resin was filtered off and the filtrate was concentrated and dried under high vacuum to give (HO)₂-[G-1]-Acet as a colorless oil. Yield: 7.87 g (97%).

### General Procedure for the Azide/Alkyne Cycloaddition Catalyzed by Cu(PPh₃)₃Br (Method B).

To a 50 mL THF solution of (An)₂-[G2]-Acet, 3.2, (5.00 g, 10.3 mmol) and (HO)₄-[G2]-N₃, 3.6, (4.83 g, 9.83 mmol) were added N,N-diisopropylethylamine (1.33 g, 10.3 mmol) and Cu(PPh₃)₃Br (19.0 mg, 206 (mol). The reaction mixture was then allowed to stir at room temperature for 12 h. LC-MS indicated the complete consumption of the azide. The solvent was evaporated and the crude product was purified by column chromatography eluting with ethylacetate and gradually increasing the polarity to MeOH:EtOAc (20:80) to give 3.9 as a colorless solid. Yield: 8.95 g (91%).

### General Procedure for the Azide/Alkyne Cycloaddition Catalyzed by CuSO₄.5H₂O and Sodium Ascorbate (Method A).

To a 20 mL THF:H₂O (3:1) solution of (An)₂-[G2]-Acet, 3.2, (5.00 g, 10.3 mmol) and (HO)₄-[G2]-N₃ 3.6 (4.83 g, 9.83 mmol) were added sodium ascorbate (306 mg, 1.55 mmol) and CuSO₄.5H₂O (129 mg, 515 (mol). The reaction mixture was then allowed to stir for 12 h at ambient temperature. The solvents were evaporated and the crude product was purified by column chromatography eluting with ethylacetate and gradually increasing the polarity to 20:80 MeOH:EtOAc to give to give 3.9 as a colorless solid. Yield: 9.33 g (95%).

### General Procedure for the Acetylene Modification of the Periphery Via the Acetylene Anhydride Coupling Reaction, (An)₂-[G-2]-[G-2]-(OH)₄.

To a 20 mL CH₂Cl₂ solution of (An)₂-[G-2]-[G-2]-(OH)₄. (5.00 g, 5.12 mmol), Pyridine (8.10 g, 102 mmol), and DMAP (375 mg, 3.07 mmol) the anhydride of pent-4-ynoic acid (4.74 g, 26.6 mmol) was added. The solution was stirred at RT over night and monitored with ¹³C NMR until the reaction reached completion (determined by the presence of the excess anhydride ˜167 ppm). The excess anhydride was quenched with 2 ml of water under vigorous stirring, followed of dilution with 300 ml of CH₂Cl₂ and the solution was extracted with 10% of NaHSO₄ (3×500 ml), and 10% of Na₂CO₃ (3×500 ml). The organic phase was dried (MgSO₄), filtered, concentrated and purified by liquid column chromatography on silica gel, eluting with hexane and gradually increasing the polarity to EtOAc:hexane (80:20) to give (Acet)₄-[G-2]-[G-2]-(An)₂ as a colorless oil. Yield: 6.04 g (91%).

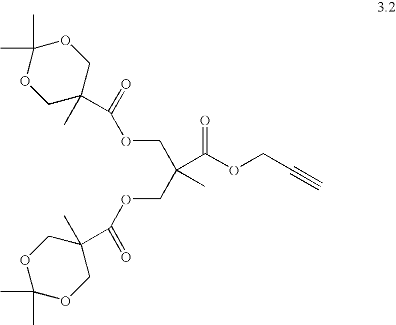

(An)₂-[G-2]-Acet, 3.2. Isolated as white solid. Yield: 25.6 g (91%). ESI MS: 486 (MH⁺).

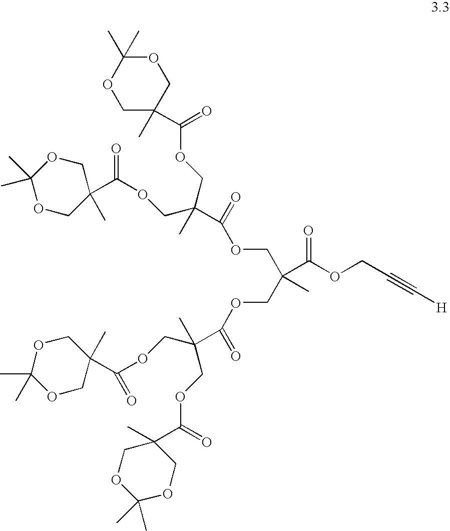

(An)₄-[G-3]-Acet, 3.3. Isolated as white solid. Yield: 20 g (81%). MALDI MS Calcd for C₅₀H₇₆O₂₂: 1028.48. Found: 1052 (MNa⁺).

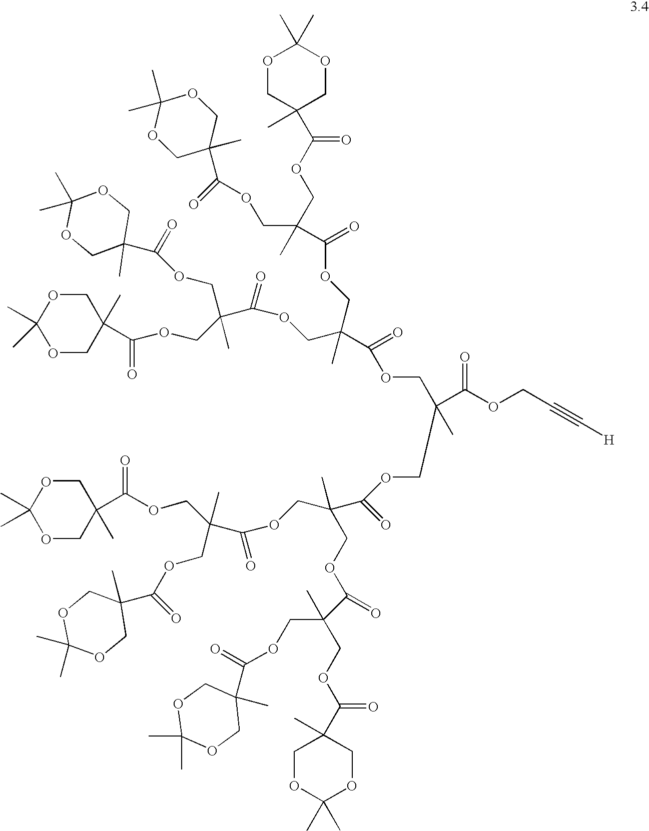

(An)₈-[G-4]-Acet, 3.4. Isolated as colorless gel. Yield: 25 g (92%). MALDI MS Calcd for C₁₀₂H₁₅₆O₄₆: 2116.99. Found: 2140 (MNa⁺). T_{g}=5° C.

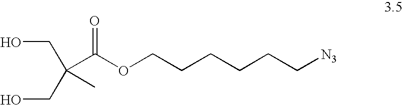

(OH)₂-[G-1]-Az, 3.5. Isolated as white solid. Yield 16.5 g (83%). ESI MS: 260 (MH⁺).

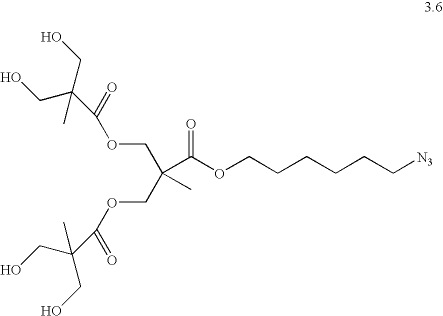

(OH)₄-[G-2]-Az, 3.6. Isolated as white solid. Yield: 15.0 g (92%). ESI MS: 493 (MH⁺).

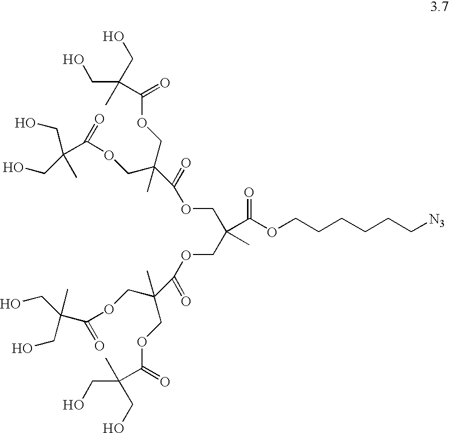

(OH)₈-[G-3]-Az, 3.7. Isolated as white solid. 15.2 g (91%). ESI MS: 957 (MH⁺).

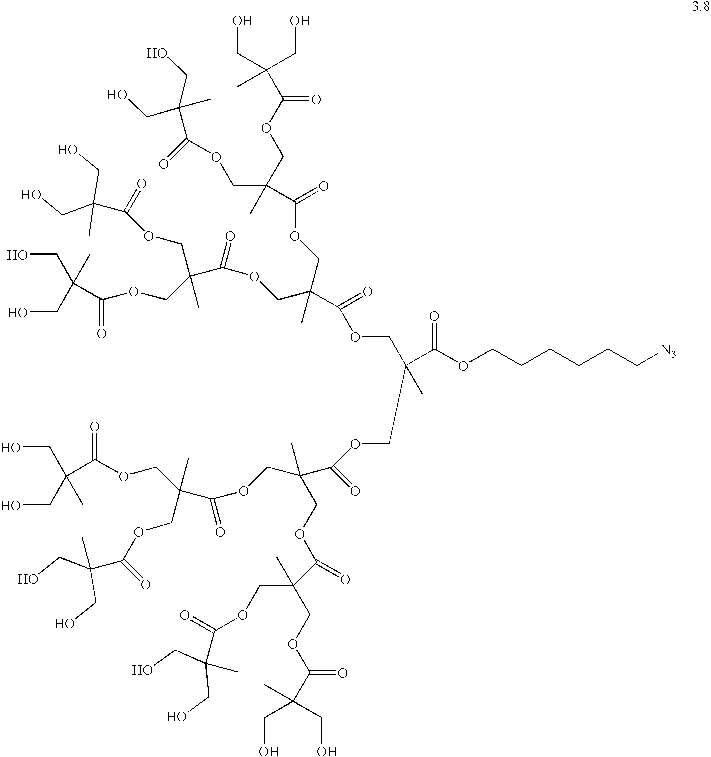

(OH)₁₆-[G-4]-Az, 3.8. Isolated as white solid. Yield: 16 g (93%). MALDI MS Calcd for C₈₁H₁₃₃N₃O₄₆: 1883.82. Found: 1907 (MNa⁺). T_{g}=57° C.

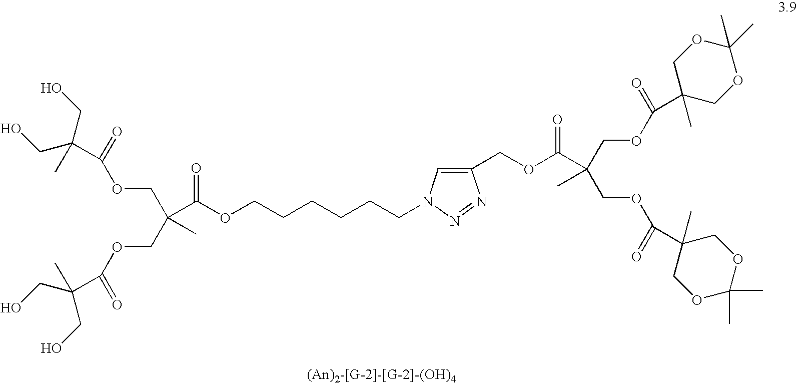

(An)₂-[G-2]-[G-2]-(OH)₄, 3.9. Isolated as white solid. Yield: 9.93 g (95%). ESI MS: 977 (MH⁺).

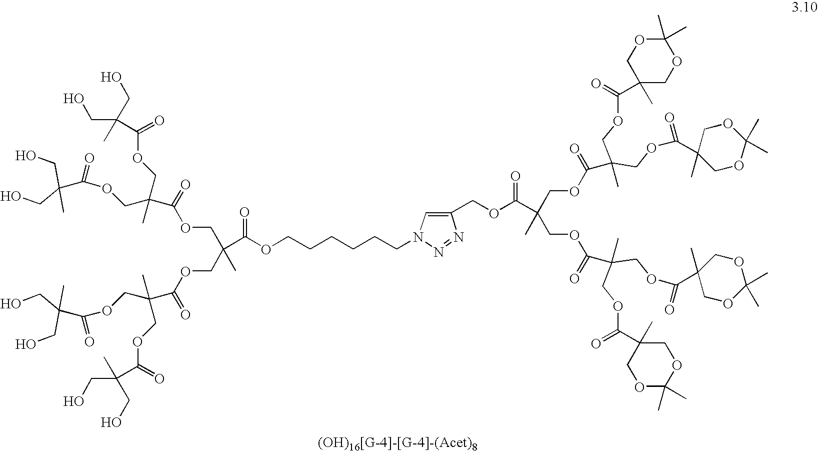

(An)₄-[G-3]-[G-3]-(OH)₈, 3.10. Isolated as white solid. Yield: 4.0 g (92%). MALDI MS Calcd for C₉₁H₁₄₅N₃O₄₄: 1983.92. Found: 1985 (MH⁺).

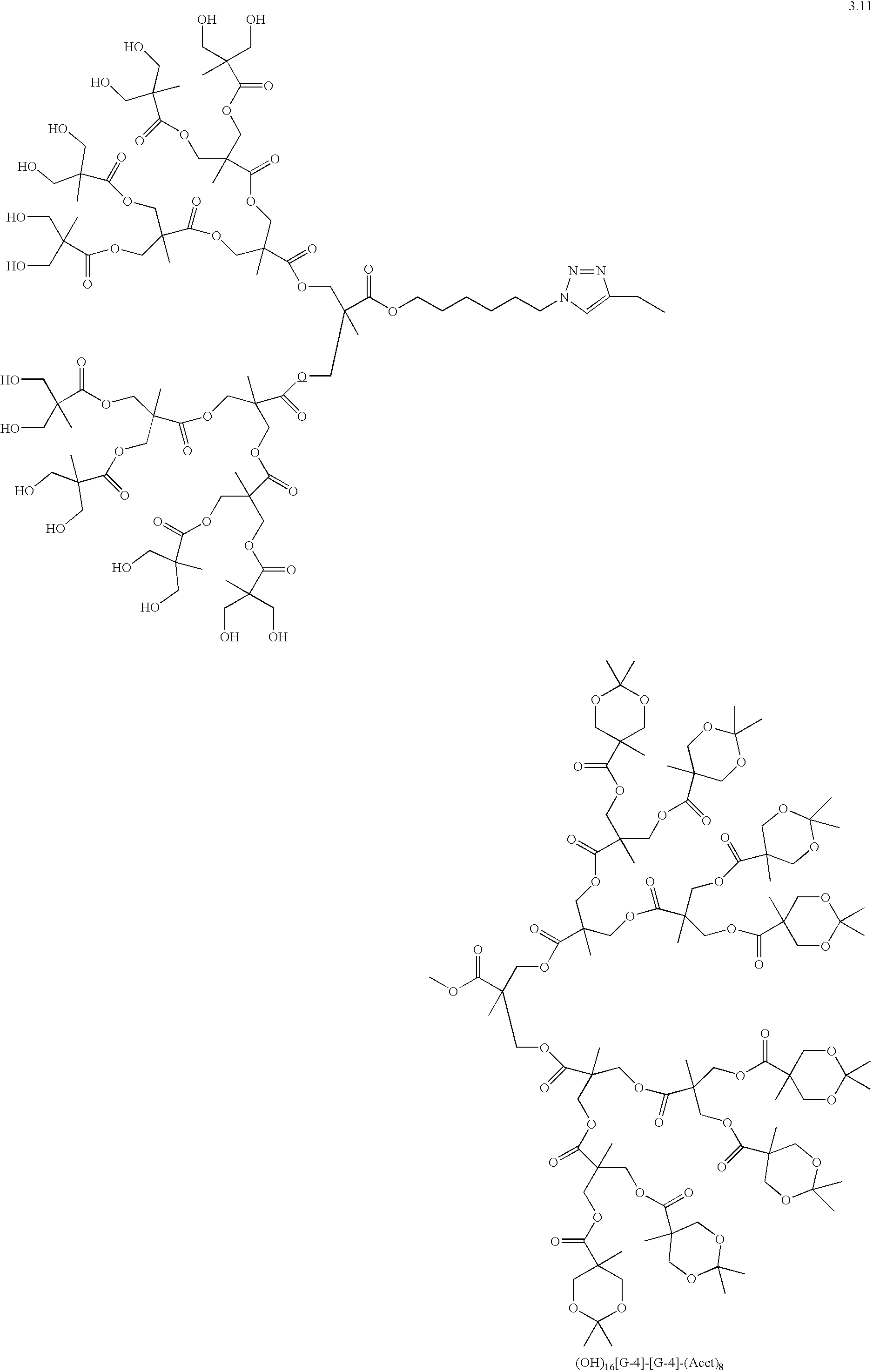

(An)₈-[G-4]-[G-4]-(OH)₁₆, 3.11. Isolated as white solid. Yield: 5.2 g (91%). MALDI MS Calcd for C₁₈₃H₂₈₉N₃O₉₂: 4000.8. Found: 4024 (Mna⁺).

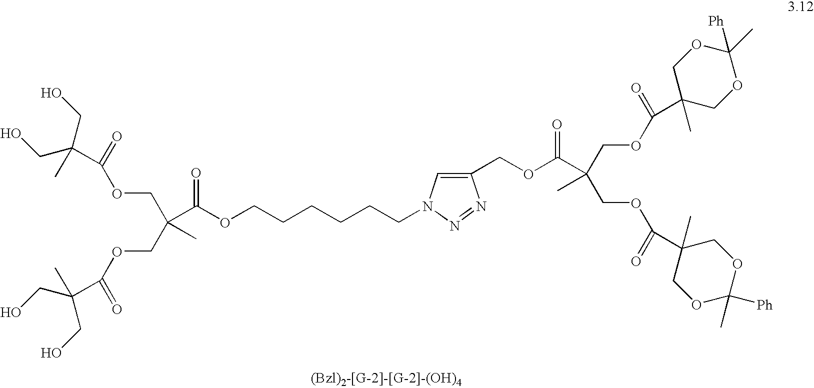

(Bzl)₂-[G-2]-[G-2]-(OH)₄, 3.12. Isolated as white solid. Yield: 1.2 g (94%). MALDI MS Calcd for C₁₅₃H₇₃N₃O₂₀: 1071.48. Found: 1073 (MH⁺), 1095 (Mna⁺).

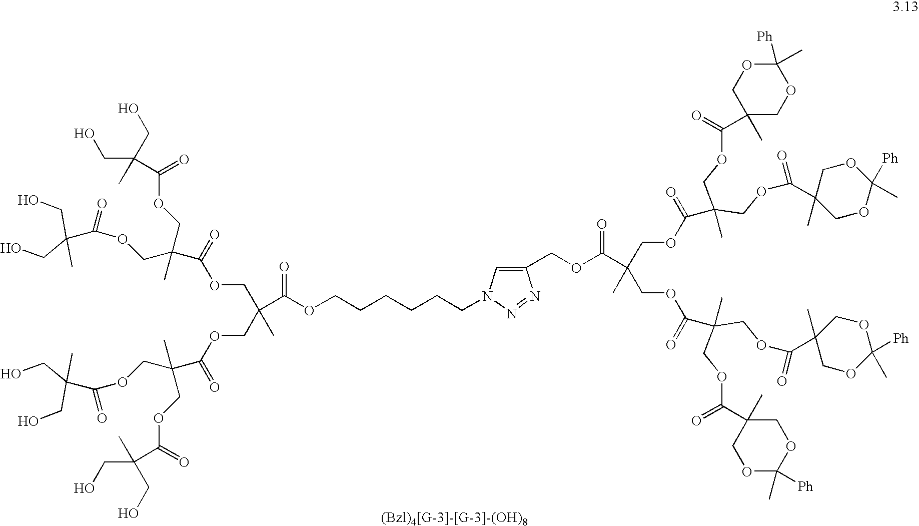

(Bzl)₄-[G-3]-[G-3]-(OH)₈, 3.13 Isolated as white solid. Yield: 1.0 g (85%). MALDI MS Calcd for C₁₀₇H₁₄₅N₃O₄₄: 2175.92. Found: 2176 (MH⁺).

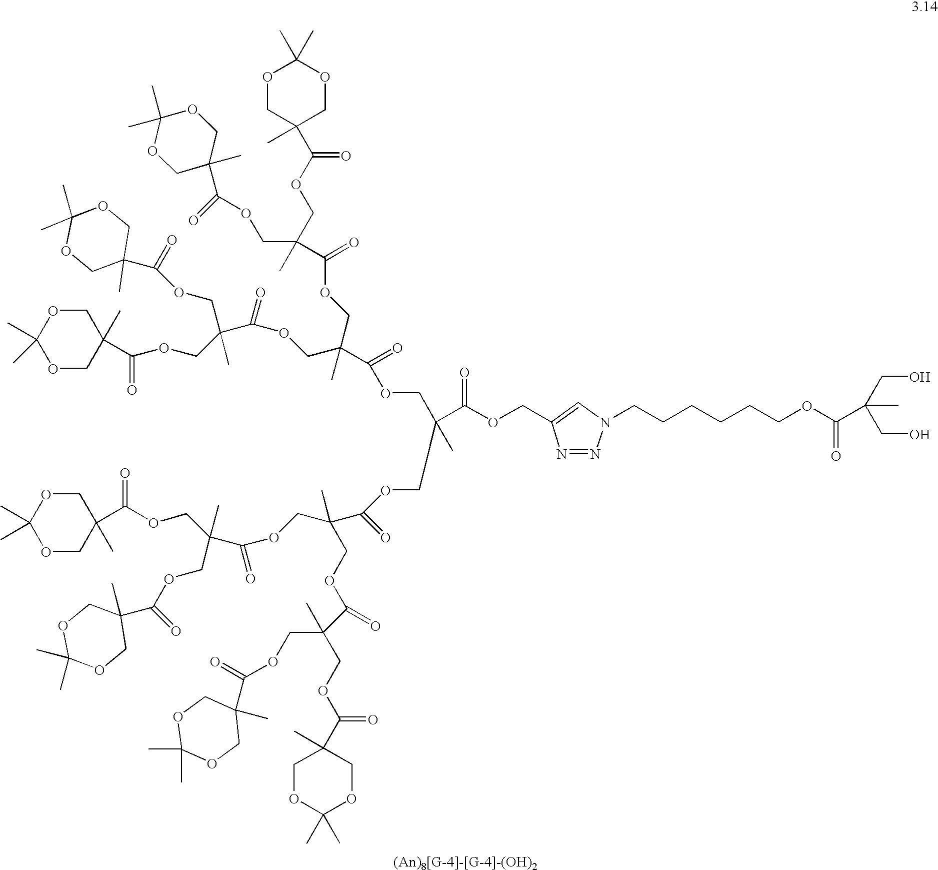

(An)₈-[G-4]-[G-4]-(OH)₂, 3.14. Isolated as colorless oil. Yield: 3.2 g (92%). MALDI MS Calcd for C₁₁₃H₁₇₇N₃O₅₀: 2376.14. Found: 2399 (MNa⁺).

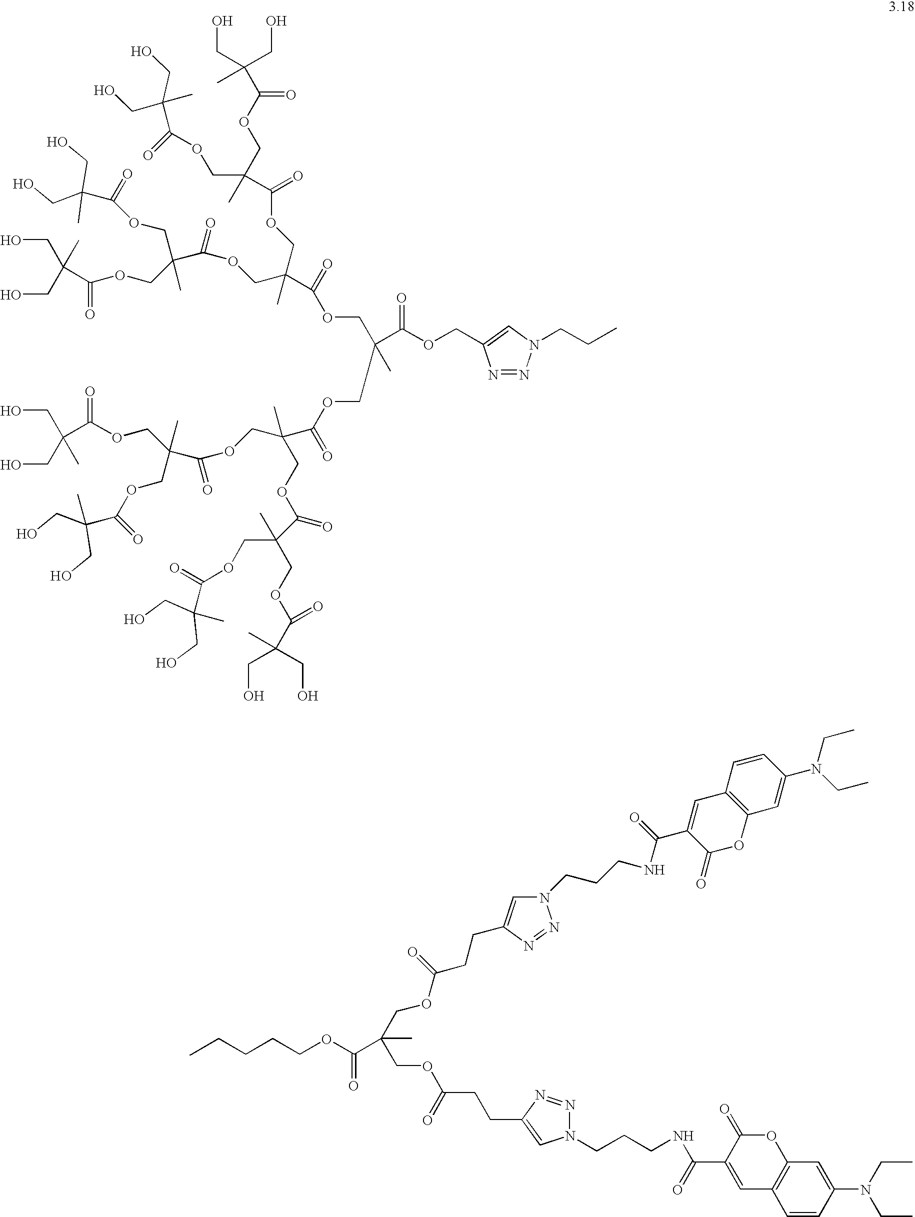

3.18. Isolated as a yellow solid. Yield; 0.89 g (91%).

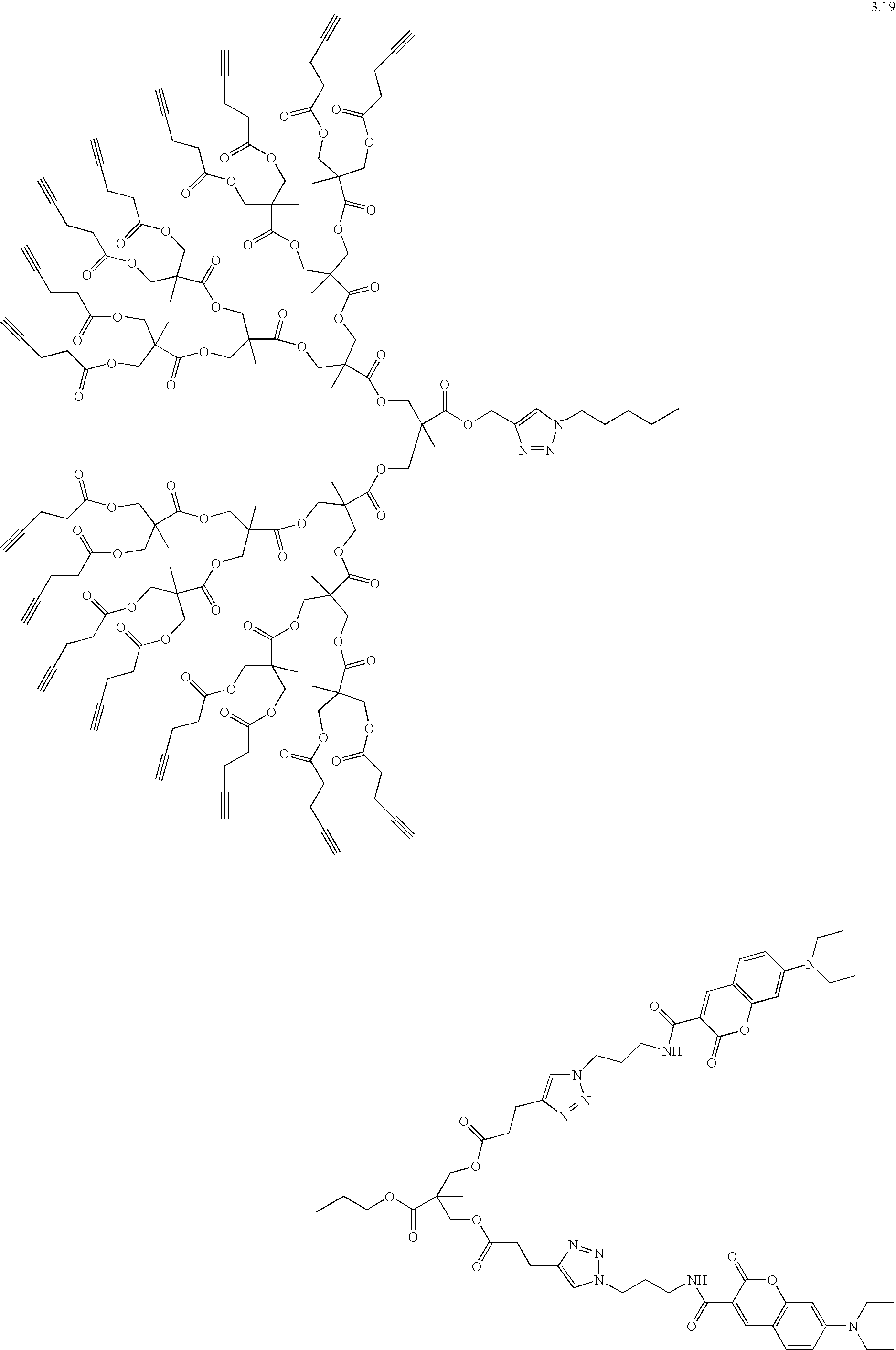

3.19. Isolated as yellow oil. Yield: 0.81 g (90%). MALDI MS Calcd for C₂₁₃H₂₅₉N₁₃O₇₄: 4182.69. Found: 4184 (MH⁺).

What is claimed is:

1. A process for making a di-block dendrimer having a first dendritic block and a second dendritic block, said first dendritic block having a first block core, said second dendritic block having a second block core, said process comprising the step of coupling the first block core to the second block core by means of a click chemistry reaction to form the di-block dendrimer having a di-block core.

2. A process according to claim 1 wherein the click chemistry reaction is a copper(I)-catalyzed 1,3-dipolar cycloaddition of a terminal acetylene with an azide to form a [1,2,3]-triazole.

3. A process according to claim 1 wherein the first block core includes a terminal acetylene and the second core block includes an azide.

4. A process according to claim 1 wherein the first dendritic block includes a first periphery, the second dendritic block includes a second periphery, and the first periphery differs from the second periphery.

5. An improved dendritic block having a block core characterized by having a terminal acetylene.

6. An improved dendritic block having a block core characterized by having an azide.

7. An improved di-block dendrimer having a first dendritic block, a second dendritic block, and a di-block core that couples the first dendritic block to the second dendritic block, the di-block core being characterized by a [1,2,3]-triazole ring that couples the first dendritic block to the second dendritic block.
